(19) United States
(12) Patent Application Publication
(10) Pub. No.: US 2006/0129215 A1
(43) Pub. Date: Jun. 15, 2006

(54) Medical devices having nanostructured regions for controlled tissue biocompatibility and drug delivery

(76) Inventors: Michael N. Helmus, Worcester, MA (US); Yixin Xu, Newton, MA (US); Shrirang V. Ranade, Arlington, MA (US)

Correspondence Address: MAYER & WILLIAMS PC, 251 NORTH AVENUE WEST, 2ND FLOOR, WESTFIELD, NJ, 07090, US

(21) Appl. No.: 11/007,867

(22) Filed: Dec. 9, 2004

(51) Int. Cl. A61N 1/05

(52) U.S. Cl. 607/115

(57) ABSTRACT

According to certain aspects of the invention, implantable or insertable medical devices are provided that contain one or more nanoporous regions, which may further comprise interconnected nanopores. Other aspects of the invention are directed to implantable or insertable medical devices that contain one or more nanostructured regions, which are formed by a variety of methods. Still other aspects of the invention are directed to implantable or insertable medical devices having nanotextured surface regions, in which cell-adhesion-promoting biomolecules (e.g., glycosaminoglycans, proteoglycans, cell adhesion peptides, and adhesive proteins) are provided on, within or beneath the nanotextured surface regions.

### TECHNICAL FIELD

This invention relates to medical devices having nanostructured regions, including nanotextured and nanoporous regions.

### BACKGROUND

It is known that nanostructured surfaces can directly interact with cell receptors, thereby controlling the adhesion or non-adhesion of cells to the surface. Furthermore, certain ceramics have been shown to be bioactive materials. A “bioactive material” is a material that promotes good adhesion with adjacent tissue, for example, bone tissue or soft tissue, with minimal adverse biological effects (e.g., the formation of connective tissue such as fibrous connective tissue). Examples of bioactive ceramic materials, sometimes referred to as “bioceramics,” include calcium phosphate ceramics, for example, hydroxyapatite; calcium-phosphate glasses, sometimes referred to as glass ceramics, for example, bioglass; and metal oxide ceramics, for example, alumina and titania.

The in-situ presentation and/or delivery of a biologically active agent within the body of a patient are common in the practice of modern medicine. In-situ presentation and/or delivery of biologically active agents are often implemented using medical devices that may be temporarily or permanently placed at a target site within the body. These medical devices can be maintained, as required, at their target sites for short or prolonged periods of time, in order to deliver biologically active agent to the target site.

### SUMMARY OF THE INVENTION

According to some aspects of the invention, implantable or insertable medical devices are provided, which contain one or more nanoporous regions having interconnected nanopores. In these aspects, a biologically active agent is disposed within the interconnected nanopores of the nanoporous region. In some embodiments, the lateral dimensions of the nanopores are controlled such that they approach the hydrated radius of the biologically active agent. In some embodiments, the biologically active agent is established within the nanoporous region concurrently with the formation of the nanoporous region, at temperatures that are less than the degradation temperature of the biologically active agent.

In accordance with other aspects of the present invention, implantable or insertable medical devices are provided, which contain one or more nanoporous regions. The nanoporous regions are formed by a method that includes the steps of: (a) providing a precursor region that comprises a first material, which is present in nano-domains within the precursor region; and (b) subjecting the precursor region to conditions under which the first material is either reduced in volume or eliminated from the precursor region, thereby forming a nanoporous region.

In accordance with other aspects of the present invention, implantable or insertable medical devices are provided, which contain one or more nanostructured regions, which are provided by a method that comprises one or more of the following processes: (a) a physical vapor deposition process comprising evaporation of a metal or a metal oxide, (b) a physical vapor deposition process comprising sublimation of a metal or ceramic material, (c) a physical vapor deposition process comprising sputtering of a metal or metal oxide, (d) a physical vapor deposition process comprising laser ablation of a metal or ceramic material, (e) simultaneous physical vapor deposition of (i) a metal or a ceramic material and (ii) a biologically active agent, (f) ion deposition of a metal or metal oxide layer, (h) ion implantation into a metal or ceramic surface, (i) X-ray lithography of a metal or ceramic surface, (j) a kinetic metallization process, (k) chemical vapor deposition of a metal or ceramic material, (l) electrodeposition and (m) electroless deposition.

In accordance with still other aspects of the present invention, implantable or insertable medical devices are provided, which comprise nanotextured surface regions. In these aspects, cell-adhesion-promoting biomolecules (e.g., glycosaminoglycans, proteoglycans, cell adhesion peptides, and adhesive proteins) are provided on, within or beneath the nanotextured surface regions.

An advantage of the present invention is that medical devices can be provided which have controlled biologic interactions.

Another advantage of the present invention is that medical devices can be provided that release biologically active agent after administration to a patient.

Yet another advantage of the present invention is that biologically active agents can be provided within nanostructured regions of medical devices using low temperature processing.

These and other embodiments and advantages of the present invention will become immediately apparent to those of ordinary skill in the art upon review of the Detailed Description and claims to follow.

### BRIEF DESCRIPTION OF THE DRAWINGS

FIG. 1 is a schematic illustration of a cylindrical pore.

### DETAILED DESCRIPTION

The present invention is directed to medical devices having one or more nanostructured regions. In some embodiments, the nanostructured regions correspond to the entire medical device or to one or more entire components of the medical device. In some embodiments, one or more nanostructured regions are disposed over or formed within a substrate surface, allowing the nanostructured regions to be provided at desired locations and in desired geometries.

As used herein, a “nanostructured” region is one that comprises numerous nanofeatures. “Features” include both geometric features (e.g., raised features, depressed features, voids, etc.) and compositional features (e.g., surface grains, material domains, etc.). Features can occur both at the surface and in the volume of the nanostructured region. A “nanofeature” is a feature having at least one dimension that is less than 100 nm in length. The nanostructured regions of the present invention will routinely contain at least 10⁶, 10⁹, 10¹² or more nanofeatures per cm² (in the case of surface nanofeatures, or per cm³ in the case of volumetric nanofeatures). Frequently, the nanostructured regions of the present invention will also contain features that are not nanofeatures (e.g., features that are larger than nanofeatures).

Where geometric nanofeatures are formed at a surface, the surface is sometimes referred to as a “nanotextured” surface. Some specific examples of surface nanofeatures include ridges, hills, mesas/plateaus, terraces, trenches, surface pores, and so forth. As a specific example, it is noted that a ridge or trench that is 10 nm wide by 50 microns long is a nanostructure, as the term is used herein, because it is has at least one dimension (e.g., its width), which is less than 100 nm in length.

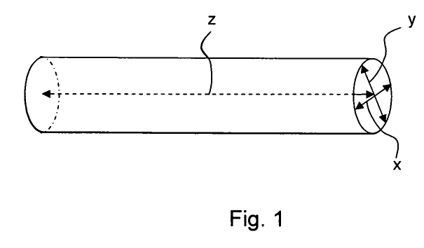

Various embodiments of the present invention are directed to medical devices containing one or more nanoporous regions. As used herein a “nanoporous region” is a volume that contains a plurality of nanopores. A “nanopore” is a void having at least one dimension that does not exceed 100 nm in length. Typically a nanopore has at least two orthogonal (i.e., perpendicular) dimensions that do not exceed 100 nm and a third orthogonal dimension, which can be greater than 100 nm. By way of example, an idealized cylindrical nanopore is illustrated in FIG. 1. Being a nanopore, the orthogonal dimensions “x” and “y” of the cylindrical pore of FIG. 1 do not exceed 100 nm in length, although the third orthogonal dimension “z” can be greater than 100 nm. As above, nanoporous regions can further comprise pores that are not nanopores.

Nanopores include surface nanopores (i.e., nanopores that extend to the surface) or sub-surface nanopores (i.e., nanopores that do not extend to the surface, unless, for example, it does so via interconnection with surface pores). In this regard, in certain embodiments, nanopores within a given nanoporous region are interconnected with each other, enhancing the ability of the nanoporous region to be used, for example, as a reservoir for the storage and delivery of biologically active agents.

Among other effects, providing medical devices with nanostructured surfaces is known to influence cellular interactions at the cell receptor level. In some embodiments, this effect is supplemented by the use of materials which are bioactive in nature. By “bioactive” is meant that these materials promote good adhesion with adjacent tissue (e.g., bone tissue, vascular tissue, mucosal tissue or soft tissue), with minimal adverse biological effects (e.g., the formation of connective tissue such as fibrous connective tissue). Examples of known bioactive materials include hydroxyapatite and oxides of titanium and aluminum. Moreover, metal oxide bioactivity has been shown to depend upon the surface nanostructure. See, e.g., Viitala R. et al., “Surface properties of in vitro bioactive and non-bioactive sol-gel derived materials,” Biomaterials. 2002 August; 23(15):3073-86.

Several aspects of the present invention concern the use of nanostructured regions for the storage, presentation and/or delivery of biologically active agents such as small molecule drugs, proteins, nucleotide sequences, and so forth. Hence, where provided, the biologically active agents are released from medical devices in some embodiments, while in other embodiments the biologically active agents remain associated with the medical devices. Biologically active agents are disposed upon or within the medical devices of the present invention for a variety of purposes including, for example, to effect in vivo release of biologically active agents (which release may be, for example, immediate or sustained), to influence (e.g., promote or inhibit) bonding between the medical device and adjacent tissue, to influence thromboresistance, to influence antihyperplastic behavior, to enhance recellularization, and to promote tissue neogenesis, among many other purposes.

Hence, in some embodiments, biologically active agents are be utilized to enhance the cellular interaction effects that arise due to the presence of nanostructured surfaces, which effects are even further enhanced in certain instances by utilizing nanostructured surfaces within bioactive materials.

In some embodiments of the present invention, including various techniques discussed herein, a biologically active agent is established within the interconnected nanopores of a nanoporous region concurrently with the formation of the nanoporous region and at low temperatures. As defined herein, “low temperatures” are temperatures less than 100° C., typically less than 60° C., and in many instances room temperature (e.g., 15-35° C.). More fundamentally, the biologically active agent is established concurrently with the nanoporous region over times and at temperatures that do not result in degradation and loss of activity of the biologically active agent.

Nanostructured regions commonly have very high surface areas associated with them. For example, it is noted that nanotextured surfaces have significantly higher surface areas as compared to corresponding flat projected surfaces. This increase in surface area can be capitalized on in various ways. For example, in some embodiments, biologically active agents are bound or adsorbed to a nanotextured surface, thereby providing higher availability of biologically active agent at the medical device surface than is obtained with a polished non-textured surface.

It is also noted that nanoporous regions have various characteristics that are driven by surface area. In this regard, as pores diameters reach nanometer-size dimensions, the surface area of the pores becomes significant with respect to the volume of the pores. As the diameter of the pore approaches the diameter of the agent to be delivered, the surface interactions dominate release rates. See, e.g., Tejal A. Desai, Derek Hansford, “Mauro Ferrari Characterization of micromachined silicon membranes for immunoisolation and bioseparation applications J. Membrane Science,” 159 (1999) 221-231, which describes insulin release through silicone nanomembranes. Furthermore, the amount of biologically active agent released and the duration of that release are also affected by the depth and tortuousity of the nanopores within the nanoporous region.

While drug delivery from nanoporous materials is known, prior drug delivery efforts have generally involved materials with parallel or near parallel pore structures that extend more or less perpendicularly to the surface. In accordance with certain aspects of the present invention, however, medical devices are provided which contain nanoporous regions with interconnected pores. In certain embodiments, the lateral dimensions (e.g., the radii) of the interconnnected nanopores approach the lateral dimensions (e.g., the hydrated radius) of the biologically active agent that is being released. The agent can move and ultimately be released from pores of these diameters, as opposed to being trapped by pores having smaller diameters. In these embodiments, the interactions between the biologically active agent and the walls of the nanopores will have a significant effect upon the release profile that is observed. Systems where the biologically active agent acts in accordance with these principles will release in a manner that is controllable and have the potential to approach zero order release kinetics. Systems where the pores are larger, on the other hand, will not control release by significant interaction between the biologically active agent with the pore wall (i.e. the majority of the biologically active agent will not interact with the pore wall at any moment in time). Release from these systems can be uncontrolled, for example, the biologically active agent will dump into the surrounding areas or at best be indicative of release through a tortuous path, as described in the percolation literature.

Nanoporous regions having interconnected pores may be formed by a variety of methods including several methods discussed herein. In some cases, the biologically active agent is introduced subsequent to the formation of the nanoporous region. In other cases and depending upon the nature of the biologically active agent and the process selected, the biologically active agent is incorporated concurrently with the formation of the nanoporous region.

In certain embodiments of the invention, nanostructured regions are formed by a method that includes: (a) providing a precursor region that comprises first and second solid materials and (b) subjecting the precursor region to conditions under which the first material is either reduced in volume or eliminated entirely from the precursor region. By providing nano-domains of the first material within the precursor region in step (a), a nanoporous region is formed in step (b). A “nano-domain” is a domain (i.e., material region) that has at least one dimension, and typically at least two orthogonal (i.e., perpendicular) dimensions, that do not exceed 100 nm in length. As above, in many instances, domains will also be present that are not nano-domains.

The above procedures can be used to create nanostructured regions having interconnected nanopores, as well as having nanotextured surfaces which are known to control biologic interactions, including tissue adhesion, as previously discussed.

Moreover, using the above procedures, (a) an entire medical device (e.g., a stent) or an entire component of a medical device may be formed, (b) one or more nanoporous regions (e.g., a coating) may be formed on a medical device substrate (e.g., the outside surface of a stent), or (c) one or more nanoporous regions may be first formed and subsequently attached to a medical device substrate.

In some embodiments of the invention, nanoporous regions are created from a mixture that contains sinterable nanoparticles and evanescent nanoparticles, with the evanescent nanoparticles forming nano-domains as described above. Once the mixture of nanoparticles is provided in the desired form (e.g., in a mold of a desired shape, in a layer adjacent a detachable surface, or in a layer adjacent a non-detachable surface such as the surface of a preexisting medical device substrate, among others), the nanoparticles are heated under conditions that are sufficient to sinter the sinterable nanoparticles and are also sufficient to reduce the volume of at least a portion of the evanescent nanoparticles, thereby forming the one or more nanoporous regions.

A “nanoparticle” is a particle (i.e., an object of regular or irregular shape including spherical, cubic, oblong, cylindrical, and other shapes) having at least one dimension, and typically at least two or even three orthogonal (i.e., perpendicular) dimensions, that do not exceed 100 nm in length.

Using sintering techniques, monolithic medical device structures (e.g., stents) are created in some embodiments, which require no subsequent shaping (e.g., no post-cutting or forming). Alternatively, nanoporous regions can be formed and subsequently shaped. Nanoporous regions can also be formed and attached to an existing medical device structure, or they can be formed on an existing medical device structure.

If desired, gradient pore volumes can be created by varying the sinterable-to-evanescent nanoparticle ratio as a function of distance, for example, the depth into the nanoporous region or the length along the medical device, to control mechanical properties, drug release characteristics, and so forth.

The sinterable nanoparticles are formed from any of a variety of materials, so long as the material is sinterable and its properties are fit for the desired medical application. Examples of such materials include suitable members of the metal, ceramic and polymer materials listed below. Specific examples of materials for this application include, for example, metals such as titanium; ceramics such as hydroxyapatite and other minerals based on calcium phosphate, bioglass, and oxides of aluminum and transition metals; and polymers such as PTFE and polyimide. As a general rule, sintering temperature decreases with decreasing particle size. Hence, sinterable nanoparticles generally have minimum sintering temperatures that are lower than those of their larger counterparts.

A variety of materials are available for use as evanescent nanoparticles, so long as at least a portion of the nanoparticles is removed during sintering, and so long as any remaining evanescent nanoparticle material or residue is not incompatible with the desired medical application. Materials for the evanescent nanoparticles include materials that are converted into gaseous species during sintering.

In some embodiments, the evanescent nanoparticles contain materials that sublime, or that melt and then evaporate, under sintering conditions. Examples of such materials include sublimable metals such as calcium and magnesium. In some instances, the sublimable metal is directly incorporated into the mixture. In other instances, the oxide form of the evanescent nanoparticle material (e.g., oxides of calcium and magnesium) are mixed with the sinterable nanoparticles (e.g., titanium), for example, to ease fabrication and improve safety. By subsequently conducting sintering in a reducing atmosphere (e.g., the atmosphere produced in a hydrogen furnace), the oxides are reduced to sublimable materials (e.g., calcium and magnesium metal), and pores are formed as the metal sublimes. Additional examples of sublimable materials include sublimable organic solids, for example, camphor or naphtha, which are particularly appropriate for use with materials that sinter at relatively low temperature, such as polymer containing nanoparticles.

In a related aspect of the invention, filled nanoporous regions are created from a mixture that contains sinterable nanoparticles and nanoparticles of biologically active agent which do not undergo significant thermal degradation at temperatures that are effective to sinter the sinterable nanoparticles. As above, these techniques are particularly appropriate for use with materials that sinter at relatively low temperature.

In other embodiments, the evanescent nanoparticles contain materials that react with gaseous species in the surrounding atmosphere during sintering and form one or more gaseous by-products. Examples include nanoparticles that react with oxygen (i.e., combust) to form gaseous by-products such as carbon dioxide and water.

In accordance with other embodiments of the invention, nanoporous regions are created from a mixture that contains two or more metals of differing nobility and (b) oxidizing and removing at least one of the less noble metals from the mixture, thereby forming a nanoporous region. In these embodiments, the at least one less noble metal corresponds to the nano-domains described above.

Various methods are available for oxidizing and removing the less noble metal(s) from the metal mixture, including (a) contact with an appropriate acid (e.g., nitric acid), (b) application of a voltage of sufficient magnitude and bias during immersion in a suitable electrolyte, and (c) heating in the presence of oxygen, followed by dissolution of the resultant oxide.

Examples include alloys of essentially any substantially non-oxidizing noble metal (e.g., gold, platinum, etc.) having nano-domains of essentially any metal that can be reacted and dissolved (e.g. Zn, Fe, Cu, Ag, etc.). Specific examples of suitable alloys include alloys comprising gold and silver (in which the silver is oxidized and removed), alloys comprising gold and copper (in which the copper is oxidized and removed), and so forth.

Further details concerning dealloying can be found, for example, in j. Erlebacher et al., “Evolution of nanoporosity in dealloying,” Nature, Vo. 410, 22 Mar. 2001, 450-453; A. J. Forty, “Corrosion micromorphology of noble metal alloys and depletion gilding,” Nature, Vol. 282, 6 Dec. 1979, 597-598; and R. C. Newman et al., “Alloy Corrosion,” MRS Bulletin, July 1999, 24-28.

Other aspects of the present invention are directed to medical devices containing one or more nanoporous regions, which are provided by a method that comprises: (a) providing a metal matrix with nanoscale metal oxide inclusions in a metal matrix and (b) subjecting the metal oxide to conditions that are sufficient to reduce the metal oxide to its corresponding metal. The reduction of the metal oxide to a pure metal is accompanied by removal of oxygen and a loss in volume, resulting in the creation of a nanoporous region Hence, in this embodiment, metal oxide inclusions that are reduced correspond to the nano-domains described above. In many embodiments, a metal oxide is selected which, after reduction, does not readily and spontaneously reform under atmospheric or physiological conditions.

For example, in some embodiments, a mixture of metal and metal oxide nanoparticles are heated under a reducing atmosphere (e.g., within a hydrogen furnace) at temperatures that are sufficiently high to both reduce the metal oxide to metal, while also sintering the metal particles into a consolidated nanoporous region. Examples of metal/metal oxides pairs include tantalum/tantalum oxide, hafnium/hafnium oxide, zironium/zirconia, and so forth. Moreover, in some embodiments, monolithic metal oxide structures are reduced under processing condition such that the structure forms nanopores, with the temperature and pressure being such that sintering occurs, while consolidation and densification do not.

Other aspects of the present invention concern the formation of nanostructured regions, including nanotextured and nanoporous regions, using sol-gel techniques. Like the above techniques, sol-gel processes can be used create an entire medical device or an entire medical device component. Moreover, sol-gel processes can be used to provide nanostructured regions on medical device substrates, for example, by either forming the nanostructured regions on the substrates, or by first forming the nanostructured regions and subsequently attaching them to the substrates.

The starting materials that are used in the preparation of sol-gel regions are frequently inorganic metal salts, metallic complexes (e.g., metal acetylacetonate complexes), or organometallic compounds (e.g., metal alkoxides). In many embodiments, the starting material is subjected to hydrolysis and polymerization (sometimes referred to as a condensation) reactions to form a colloidal suspension, or “sol”.

For example, in some embodiments, an alkoxide of a metal of choice, such as a methoxide, ethoxide, isopropoxide, tert-butoxide, etc. of titanium, zirconium, hafnium, tantalum, molybdenum, tungsten, rhenium, iridium, aluminum, etc., is dissolved in a suitable solvent, for example, in one or more alcohols. Subsequently, a sol is formed, for example, by adding water or another aqueous solution, such as an acidic aqueous solution (which aqueous solution can further contain an organic solvent species such as alcohols), causing hydrolysis and polymerization. If desired, agents can be added to control the viscosity and/or surface tension of the sol.

As another example, in some embodiments, hydrated inorganic metal salts are first dissolved in a solvent, followed by the addition of a proton scavenger, which induces gel formation. In these embodiments, the proton scavenger reacts with hydrogen from the hydrated-metal species, which then undergo hydrolysis and condensation reactions to form a sol. See e.g., U.S. Application No. 20020104599.

Further processing of the sol enables ceramic materials to be made in a variety of different forms. For instance, thin films can be produced on a substrate, for example, by spray coating, coating with an applicator (e.g., by roller or brush), spin-coating, or dip-coating of the sol onto the substrate, whereby a wet gel is formed. Where dip coating is employed, the rate of withdrawal from the sol can be varied to influence the properties of the film. Monolithic wet gels can be formed, for example, by placing the sol into or onto a mold or another form (e.g., a sheet) from which the dried final product can be released.

The wet gel is then dried. If the solvent in the wet gel is removed under supercritical conditions, a highly porous material commonly called an “aerogel” is obtained. If the gel is dried via freeze drying (lyophilization), the resulting material is commonly referred to as a “cryogel.” Drying at ambient temperature and ambient pressure leads to what is commonly referred to as a “xerogel.” Other drying possibilities are available including elevated temperature drying (e.g., in an oven), vacuum drying (e.g., at ambient or elevated temperatures), critical point drying, and so forth.

The porosity of the gel can be regulated in a number of ways, including, for example, varying the solvent/water content, varying the aging time (e.g., the time before addition of an aqueous solution to a metal organic solution), varying the drying method and rate, and so forth.

In some embodiments, a biologically active agent is added to the sol prior to processing the same into a gel. In other embodiments, a biologically active agent is incorporated into or onto the gel region subsequent to the formation of the same using techniques such as those described below.

In certain embodiments, sol-gel processing is carried out at low temperatures (e.g., temperatures of 15-35° C.). This aspect of the present invention permits the incorporation of temperature sensitive agents during the course sol-gel processing.

In othr embodiments, the sol-gel is subjected to high temperatures, for example, temperatures of 100° C., 200° C., 300° C., 400° C., 500° C., or more. Such high temperatures commonly reduce the porosity of the sol-gel, while at the same time increasing its mechanical strength. Where the biologically active agent is present at high temperatures, care should be taken to avoid thermal damage to the same.

Further information concerning sol-gel materials can be found, for example, in Viitala R. et al., “Surface properties of in vitro bioactive and non-bioactive sol-gel derived materials,” Biomaterials. 2002 August; 23(15):3073-86; Radin, S. et al., “In vitro bioactivity and degradation behavior of silica xerogels intended as controlled release materials,” Biomaterials. 2002 August; 23(15):3113-22; Nicoll S. B., et al., “In vitro release kinetics of biologically active transforming growth factor-beta 1 from a novel porous glass carrier,” Biomaterials. 1997 June; 18(12):853-9; Santos, E. M. et al., “Sol-gel derived carrier for the controlled release of proteins,” Biomaterials. 1999 September; 20(18): 1695-700; Radin, S. et al., “Silica sol-gel for the controlled release of antibiotics. I. Synthesis, characterization, and in vitro release,” J Biomed Mater Res. 2001 November; 57(2):313-20; Aughenbaugh, W. et al., “Silica sol-gel for the controlled release of antibiotics. II. The effect of synthesis parameters on the in vitro release kinetics of vancomycin,” J Biomed Mater Res. 2001 Dec. 5; 57(3):321-6; Santos, E. M. et al., “Si—Ca—P xerogels and bone morphogenetic protein act synergistically on rat stromal marrow cell differentiation in vitro,” J Biomed Mater Res. 1998 July; 41(1):87-94.

Other aspects of the present invention are directed to the formation of nanostructured regions using methods that comprise physical vapor deposition, ion deposition, ion implantation, and/or X-ray lithography. These processes are typically conducted in the presence of a substrate, which can be, for example, a metal, semiconductor, ceramic or polymer substrate.

Physical vapor deposition, ion deposition, ion implantation, and X-ray lithography are frequently carried out under vacuum (i.e., at pressures that are less than ambient atmospheric pressure). By providing a vacuum environment, the mean free path between collisions of vapor particles (including atoms, molecules, ions, etc.) is increased, and the concentration of gaseous contaminants is reduced, among other effects.

Physical vapor deposition (PVD) processes are processes in which a source of material, typically a solid material, is vaporized, and transported to a substrate where a film (i.e., a layer) of the material is formed. PVD processes are generally used to deposit films with thicknesses in the range of a few nanometers to thousands of nanometers, although greater thicknesses are possible. PVD can take place in a wide range of gas pressures, for example, commonly within the range of 10⁻⁵ to 10⁻⁹ Torr. In many embodiments, the pressure associated with PVD techniques is sufficiently low such that little or no collisions occur between the vaporized source material and ambient gas molecules while traveling to the substrate. Hence, the trajectory of the vapor is generally a straight (line-of-sight) trajectory.

Some specific PVD methods that are used to form nanostructured regions in accordance with the present invention include evaporation, sublimation, sputter deposition and laser ablation deposition.

For instance, in some embodiments, a source material is evaporated or sublimed, and the resultant vapor travels from the source to a substrate, resulting in a deposited layer on the substrate. Examples of sources for these processes include resistively heated sources, heated boats and heated crucibles, among others.

Sputter deposition is another PVD process, in which surface atoms or molecules are physically ejected from a surface by bombarding the surface (commonly known as a sputter target) with high-energy ions. As above, the resultant vapor travels from the source to the substrate where it is deposited. Ions for sputtering can be produced using a variety of techniques, including arc formation (e.g., diode sputtering), transverse magnetic fields (e.g., magnetron sputtering), and extraction from glow discharges (e.g., ion beam sputtering), among others. One commonly used sputter source is the planar magnetron, in which a plasma is magnetically confined close to the target surface and ions are accelerated from the plasma to the target surface.

In accordance some embodiments of the invention, two or more materials are co-deposited using any of several PVD processes, including evaporation, sublimation, laser ablation and sputtering. For instance, two or more materials can be co-sputtered (e.g., by sputtering separate targets of each of the materials or by sputtering a single target containing multiple materials). By co-sputtering two immiscible metals, for example, an alloy film can be formed, which is then annealed to cause phase separation and the creation of a nanostructured region having a phase domain of one metal (e.g., a matrix phase) and a separate phase domain of the other metal (e.g., a disperse phase). If desired, one metal (e.g., the nano-domains corresponding to the disperse phase) can be removed preferentially, for instance, using techniques such as those discussed above, thereby producing a nanoporous region. As another example, by co-sputtering magnetic and insulating materials, magnetic nanoparticles (e.g., Fe nanoparticles) are formed in an insulating matrix (e.g., a ceramic matrix).

In some embodiments of the invention, nucleation and growth of nanoparticles in the vapor phase prior to deposition on a substrate is achieved by sputtering at higher pressures. Moreover, in some embodiments, phase separated films from thermodynamically miscible materials are created by alternatively sputtering at low and high pressures.

Further information regarding sputtering of nanostructured films can be found in Handbook of Nanophase and Nanostructured Materials. Vol. 1. Synthesis. Zhong Lin Wang, Yi Liu, and Ze Zhang, Editors; Kluwer Academic/Plenum Publishers, Chapter 9, “Nanostructured Films and Coating by Evaporation, Sputtering, Thermal Spraying, Electro- and Electroless Deposition”.

Laser ablation deposition is another PVD process, which is similar to sputter deposition, except that vaporized material is produced by directing laser radiation (e.g., pulsed laser radiation), rather than high-energy ions, onto a source material (typically referred to as a target). The vaporized source material is subsequently deposited on the substrate.

As with other PVD processes, two materials may be co-deposited (e.g., by ablating separate targets or by ablating a single target containing a combination of materials). Moreover, in some embodiments, nucleation and growth of nanoparticles in the vapor phase prior to deposition on a substrate is achieved by ablation at higher pressures.

Because many PVD processes are low temperature processes, a thermally sensitive biologically active agent can be simultaneously co-deposited with another material (e.g., a ceramic, metallic or polymeric material), for example, using techniques such as the evaporation, sublimation, sputter deposition and laser ablation techniques described above.

In still other embodiments of the present invention, nanostructured regions are produced by ion deposition processes. An “ion deposition process” is a deposition process in which ions are accelerated by an electric field, such that the substrate is bombarded with ions during the deposition process.

In some instances, the substrate is bombarded with ions during the course of a PVD deposition process to a achieve a nanostrcutred region, in which case the technique is sometimes referred to as ion beam assisted deposition. For example, the substrate can be bombarded with ions of a reactive gas such as oxygen or nitrogen, or an inert gas such as argon, during the course of a PVD process like those discussed above. These ions can be provided, for example, by means of an ion gun or another ion beam source.

In some instances, at least a portion of the deposition vapor itself is ionized and accelerated to the substrate. For example, the deposition vapor can correspond to the material to be deposited (e.g., where a vapor produced by a PVD processes such as evaporation, sublimation, sputtering or laser ablation is ionized and accelerated to the substrate). As another example, the deposition vapor can correspond to a chemical precursor of the deposited material (e.g., where a precursor vapor for a chemical vapor deposition process such as low-pressure or plasma-enhanced chemical vapor deposition is ionized and accelerated to the substrate).

Deposition vapors can be ionized using a number of techniques. For example, deposition vapor can be at least partially ionized by passing the same through a plasma. As another example, partially ionized vapor can be directly generated at a material source, for instance, by subjecting the material source to an electronic beam and/or to an arc erosion process, such as a cathodic or an anodic arc erosion processes. Specific examples of such processes include rod cathode arc-activated deposition (RAD), spotless arc deposition (SAD), and hollow cathode activated deposition (HAD).

In yet other embodiments of the invention, nanostructured regions are established by subjecting an ionic species to an electric field that is sufficiently high such that the impacting ions are implanted in or beneath the substrate surface. Such “ion implantation” processes are used, for example, to create nanoclusters of a variety of materials, including metal and ceramic materials. Suitable species for ion implantation include, for example, ionic species corresponding to an element or molecule found in the substrate, ionic species corresponding to other elements or molecules not found in the substrate, including ionic species corresponding to reactive and non-reactive species (e.g., a reactive gas such as oxygen or an inert gas such as argon).

In some cases, multiple deposition techniques are combined to form nanostructured regions on medical devices. One specific example is the deposition of polymers (e.g., by plasma enhanced polymerization) concurrently with PVD-type deposition of metals to produce mixed metal-polymer films. See “Plasma Polymer-Metal Composite Films,: H. Biedermann and L. Nartinu, p. 269 in Plasma Deposition, Treatment and Etching of Polymers, Riccardo d'Agostino, Ed., Academic Press (1990). In another specific example, ion deposition is combined with ion implantation in a process known as plasma ion immersion implantation and deposition.

In still other embodiments, nanostructured regions are established via X-ray lithography. One process, known as columnated plasma lithography, is capable of producing X-rays for lithography having wavelengths on the order of 10 nm. Once a suitable mask is provided on a substrate using X-ray lithography, the substrate is subjected to a subsequent etching, deposition or reaction step, resulting in a nanostructured surface on the substrate.

In still other embodiments of the present invention, nanostructured regions are provided on implantable or insertable medical device substrates by processes comprising a technique commonly referred to as “kinetic metallization.” In the kinetic metallization technique, metal particles (e.g., metal nanoparticles) are impacted with a substrate at high speed (e.g., at supersonic or near supersonic velocities) and at a temperature that is well below the melting point(s) of the metal particles (e.g., at a low temperature, such as ambient temperature). In certain embodiments, the metal particles are mixed with a relatively inert gas such as helium and/or nitrogen in a powder fluidizing unit, and the resulting fluidized powder is sprayed at high velocity onto the substrate. When the particles strike the substrate, fresh active metal is exposed, leading to adhesive and cohesive metallurgical bonding of the metal particles with the substrate and with one another.

Because the particles are deposited at well below their respective melting points, the particles remain solid. Hence, like many of the above deposition techniques, they can form mixtures of metals that may be immiscible as liquids. Moreover, heat distortion of the substrate and interdiffusion of multi-layer coatings can be minimized or avoided. Additional information on this process can be found, for example, in U.S. Pat. Nos. 5,795,626 and 6,074,135, U.S. Patent Application Nos. 2002/0168466 A1 and 2003/0006250 A1, and International Publication Number WO 02/085532 A1, all to Howard Gabel and Ralph Tapphorn.

The metal particles in this technique are, for example, particles of a pure metal, particles of a metal alloy, a mixture of pure metal particles, a mixture alloy particles, and so forth. Examples of particles for use in these methods include particles of the various metals described herein, including particles of aluminum, cobalt, titanium, niobium, zinc, copper, tungsten, nickel, chromium, iron, as well as alloys based on these and other metals, such as stainless steel. These and other particles can be used coat metal substrates (e.g., aluminum, titanium, stainless steel and nitinol substrates), as well as semiconductor, ceramic and polymer substrates, for example, those formed from the materials described herein.

In some embodiments, due to the ability to operate the kinetic metallization at moderate temperatures, the substrate is simultaneously co-coated with a thermally sensitive biologically active agent.

In embodiments where a nanostructured surface containing a mixture of metal nanoparticles is formed, one metal can be preferentially removed using techniques such as those discussed above, thereby producing a nanoporous coating.

In some embodiments, a metallic nanostructured surface is subjected to an oxidation process, for example, to form a ceramic oxide coating.

Other aspects of the invention involve the use of chemical vapor deposition (CVD) to produce nanostructured regions or nanoparticles. CVD is a process whereby atoms or molecules are deposited in association with a chemical reaction (e.g., a reduction reaction, an oxidation reaction, a decomposition reaction, etc.) of vapor-phase precursor species. When the pressure is less than atmospheric pressure, the CVD process is sometimes referred to as low-pressure CVD or LPCVD. Plasma-enhanced chemical vapor deposition (PECVD) techniques are chemical vapor deposition techniques in which a plasma is employed such that the precursor gas is at least partially ionized, thereby reducing the temperature that is required for chemical reaction.

A variety of materials can be formed using CVD (including LPCVD). For example, metals can be formed using metallorganic precursors or by the reduction of metal chlorides with hydrogen. As other examples, ceramics can be formed from oxygen-containing metallic precursors, or from metallic precursors (e.g., WF₆ or TiCl₄) in the presence of oxygen or an oxygen containing species. As with CVD, a wide range of materials can be deposited with PECVD. As a specific example, monomeric precursors are frequently deposited as polymer layers using PECVD.

In some CVD processes, vapor generated from solid sources (for example, using processes like those discussed above in connection with PVD), are reacted with another species (for example, a reactive gas or another vaporized solid material) in the deposition environment. As one specific example, metal ceramics can be formed by vaporizing and depositing metal in the presence of oxygen gas at low pressure.

Several of the techniques described herein rely on the use of particles to form nanostructured regions, including nanoporous regions. Particles of numerous materials, including nanoparticles, are commercially available from a number of sources. Nanoparticles are made using various techniques, including chemical vapor deposition (CVD) and chemical vapor condensation (CVC), which are particularly useful for the formation of metallic oxide nanoparticles.

In particle formation using CVD, gas phase nucleation and growth are controlled, typically by controlling the number of nuclei formed in the CVD reactor and by controlling the concentration of the condensing species in the gas phase. For example, supersaturation of the gas phase is frequently achieved by increasing the temperature and pressure in the reactor, while decreasing the flow rate. In particle formation using CVC, on the other hand, particles are also formed based on gas phase nucleation. In this process, metallorganic compounds are frequently used as precursor chemicals. For example, a carrier gas is bubbled through the precursor and the resulting vapor phase is introduced into a vacuum chamber, after which the metallorganic compounds pass through a heated zone. While in the heated zone the compounds begin to decompose thermally, and they begin to coalesce, thereby forming small clusters of particles. After passing though the heated zone, rapid expansion of the stream moderates particle growth and agglomeration. The particles are then condensed on a cooled surface and collected. Further information can be found in Handbook of Nanophase and Nanostructured Materials. Vol. 1. Synthesis. Zhong Lin Wang, Yi Liu, and Ze Zhang, Editors; Kluwer Academic/Plenum Publishers, 2003, Chapter 5, “Chemical Vapor Deposition”.

Other aspects of the present invention are directed to the formation of nanostructured regions, including nanoporous regions, using methods that comprise CVD. These processes are typically conducted in the presence of a substrate, which can be, for example, a metal, semiconductor, ceramic or polymer substrate. Unlike physical vapor deposition processes above, chemical vapor deposition processes are not necessarily line-of-sight processes, allowing coatings to be formed on substrates of complex geometry.

For example, in a process known as particle-precipitation-aided chemical vapor deposition (PP-CVD), an aerosol of particles is first formed by a gas phase reaction at elevated temperature. The particles are then deposited on a substrate, for example, due to the forces of electrophoresis, thermophoresis, or forced flow. In certain embodiments, a heterogeneous reaction occurs simultaneously with deposition to interconnect the particles and form a nanoporous layer, or the deposited particles are sintered to form a nanoporous layer, or both. As a specific example, a CO₂ laser can be used to heat metallorganic precursor compounds in the gas phase, resulting in decomposition of the precursor with concomitant formation of an aerosol of ceramic nanoparticles. The particles are then deposited on a substrate as a result of a thermal gradient that naturally exists between the heated reaction zone created by the laser and the cooler substrate. In this example, heterogeneous reactions at the substrate surface can be controlled independently of the gas phase reactions. Further information can be found in Handbook of Nanophase and Nanostructured Materials. Vol. 1. Synthesis. Zhong Lin Wang, Yi Liu, and Ze Zhang, Editors; Kluwer Academic/Plenum Publishers, Chapter 5, “Chemical Vapor Deposition”.

Nanoporous polymer films can also be deposited by CVD. For example, in hot-filament CVD (HFCVD, also known as pyrolytic or hot-wire CVD), a precursor gas is thermally decomposed by a resistively heated filament. The resulting pyrolysis products then adsorb onto a substrate maintained at around room temperature and react to form a film. For example, fluorocarbon films can be made using hexafluororpropylene oxide as a precursor gas. Due to the nucleation and growth mechanisms in the HFCVD processes, nanoporous films can be made using HFCVD. For further information, see, e.g., United States Patent Application No. 2003/0138645 to Gleason et al. and K. K. S. Lau et al., “Hot-wire chemical vapor deposition (HWCVD) of fluorocarbon and organosilicon thin films,” Thin Solid Films, 395 (2001) pp. 288-291.

In other embodiments, nanostructures are grown within preexisting porous layers using atomic-layer chemical vapor deposition. See, e.g., See Marian Nanu, “Nanostructured TiO₂—CuInS₂ based solar cells,” E-MRS Spring Meeting 2003, Jun. 10-13, 2003, SYMPOSIUM D, Thin Film and Nano-Structured Materials for Photovoltaics, Abstract No. D-X.2, in which CuInS₂ is applied inside the pores of nanoporous TiO₂, which comprises 10 to 50 nm particles, using atomic layer chemical vapor deposition (ALCVD). In this particular gas-phase deposition technique, reactants are supplied sequentially to avoid clogging of the nanopores.

Still other aspects of the present invention are directed to the formation of nanostructured regions using methods that comprise electrodeposition and/or electroless deposition. Like many of the above-described processes, these processes are typically conducted in the presence of a substrate, which can be, for example, a metal, semiconductor, ceramic or polymer substrate. Moreover, like CVD processes, these processes are desirable in some instances, because they are not necessarily line-of-sight processes. For instance, films can be deposited on and within nanoporous substrates, thereby producing corresponding nanoporous posited regions. Furthermore, because they can be conducted at low processing temperatures (e.g., at room temperature) the biologically active agents may be co-deposited and undesirable chemical reactions that occur at high temperatures (e.g., the degradation of thermally sensitive biologically active agents) are avoided.

Electrodeposition involves the application of an electric current through an ion-containing solution. During electrodeposition, positively charged ions are attracted to a negatively charged electrode (i.e., the cathode), e.g., a medical device substrate, while negatively charged ions are attracted to a positively charged electrode (i.e., the anode). The charged ions are then electrically neutralized at the electrodes, and the products of this neutralization process appear at the electrodes. Aqueous and non-aqueous electrolytes may be used, with aqueous electrolytes being more commonly used because they are good solvents for salts and because water is inexpensive. A variety of processing parameters such as electrolyte composition, pH, temperature, agitation, applied potential, current distribution and so forth can be varied to influence the characteristics of the electrodeposited coatings, including composition, thickness, nanostructure and so forth. Where a non-conductive substrate (e.g., a polymer or a ceramic substrate) is utilized, the substrate can be rendered conductive, for example, using an electroless deposition process (see below). Further information on electrodeposition can be found in Handbook of Nanophase and Nanostructured Materials. Vol. 1. Synthesis. Zhong Lin Wang, Yi Liu, and Ze Zhang, Editors; Kluwer Academic/Plenum Publishers, Chapter 5, “Chemical Vapor Deposition”.

A variety of nanostructured films can be formed by electrodeposition, including metallic, ceramic, and polymeric films. Where a metallic film is formed, the film is oxidized in certain embodiments to form a ceramic surface.

Furthermore, nanostructured regions can be formed by incorporating suspended nanoparticles into a matrix that is formed by electrodeposition. For example, nanoparticles can be dispersed by adsorbing cations on the surface of the same. During electrodeposition, the nanoparticles with adsorbed cations travel to the cathode where electrodeposition takes place, thereby incorporating the nanoparticles into the deposited layer.

Filled and unfilled nanoporous regions can be formed using such techniques. For example, in some embodiments, a nanoparticles are incorporated into an electrodeposited layer which are subsequently reduced in volume or eliminated (e.g., a sublimable, evaporable, combustible or dissolvable material such as those discussed above). In other embodiments, nanoparticles of a biologically active agent are incorporated into an electrodeposited layer.

Electroless deposition is different from electrodeposition in that electrons are produced without the need for an external current. As a result, the substrate need not be conductive. Examples of electroless deposition processes including deposition by ion exchange or charge exchange, deposition by contact with a metal to be plated, autocatalytic deposition onto catalytic surfaces in solutions containing reducing agents, and so forth. For autocatalytic deposition, a surface to be coated is treated with a catalyst. For example, the substrate can be coated with a metal catalyst which, upon exposure to a plating bath containing a reducing agent and metal ions or metal complexes, catalyzes metal deposition at the substrate surface.

As with electrodeposition above: (a) a variety of films can be formed by electroless deposition, including metallic, ceramic, and polymeric films; (b) metallic films, where formed, can be oxidized in some embodiments to form a ceramic (metal oxide) surfaces, (c) suspended nanoparticles, can be incorporated into a matrix that is formed by electroless deposition, and (d) films can be deposited on and within preexisting nanoporous substrates. As one example, see, F. Schlottig et al., “Characterization of nanoscale metal structures obtained by template synthesis,” Fresenius J Anal. Chem. (1998) 361:684-686, in which metals are autocatalytically deposited into nanometer-wide parallel pores of porous anodic oxide films on aluminum.

Hence, using the above and other techniques, nanostructured regions can be formed from a wide range of materials, including suitable materials selected from the metals, ceramics and polymers listed below.

Ceramic materials include, for example, calcium phosphate ceramics (e.g., hydroxyapatite); calcium-phosphate glasses, sometimes referred to as glass ceramics (e.g., bioglass); metal oxides, including non-transition metal oxides (e.g., oxides of metals from groups 13, 14 and 15 of the periodic table, including, for example, aluminum oxide) and transition metal oxides (e.g., oxides of metals from groups 3, 4, 5, 6, 7, 8, 9, 10, 11 and 12 of the periodic table, including, for example, oxides of titanium, zirconium, hafnium, tantalum, molybdenum, tungsten, rhenium, iridium, and so forth); and carbon based ceramic-like materials such as silicon carbides and carbon nitrides.

As noted above, nanostructured surfaces are known to directly interact with cell receptors, thereby increasing adhesion of cells to the surface. In some embodiments, this effect is supplemented by the use of materials which are bioactive in nature. By “bioactive” is meant that these materials promote good adhesion with adjacent tissue (e.g., bone tissue, vascular tissue, mucosal tissue, or soft tissue), with minimal adverse biological effects (e.g., the formation of connective tissue, more particularly, fibrous connective tissue). Examples of bioactive ceramic materials, sometimes referred to as “bioceramics,” include calcium phosphate ceramics, for example, hydroxyapatite; calcium-phosphate glasses, sometimes referred to as glass ceramics, for example, bioglass; and metal oxide ceramics, for example, alumina and titania.

Metals include, for example, silver, gold, platinum, palladium, iridium, osmium, rhodium, titanium, tungsten, and ruthenium and metal alloys such as cobalt-chromium alloys, nickel-titanium alloys (e.g., nitinol), iron-chromium alloys (e.g., stainless steels, which contain at least 50% iron and at least 11.5% chromium), cobalt-chromium-iron alloys (e.g., elgiloy alloys), and nickel-chromium alloys (e.g., inconel alloys), among others.

Polymers include, for example: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydoxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, polystyrenes, styrene-maleic anhydride copolymers, styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; ionomers; polyalkyl oxide polymers and copolymers including polyethylene oxides (PEO); glycosaminoglycans; polyesters including polyethylene terephthalates and aliphatic polyesters such as polymers and copolymers of lactide (which includes lactic acid as well as d-, l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), poly-4-methyl-pen-1-enes, ethylene-alpha-olefin copolymers, ethylene-methyl methacrylate copolymers and ethylene-vinyl acetate copolymers; polyolefin elastomers (e.g., santoprene), ethylene propylene diene monomer (EPDM) rubbers, fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as blends and further copolymers of the above.

Such polymers may be provided in a variety of configurations, including cyclic, linear and branched configurations. Branched configurations include star-shaped configurations (e.g., configurations in which three or more chains emanate from a single branch point), comb configurations (e.g., graft polymers having a main chain and a plurality of branching side chains), and dendritic configurations (e.g., arborescent and hyperbranched polymers). The polymers can be formed from a single monomer (i.e., they can be homopolymers), or they can be formed from multiple monomers (i.e., they can be copolymers) that can be distributed, for example, randomly, in an orderly fashion (e.g., in an alternating fashion), or in blocks.

In embodiments of the invention in which a nanostructured region is formed in or on an underlying substrate or is attached to an underlying substrate, the substrate material is typically a ceramic, metal or polymeric substrate, which can comprise suitable materials selected from those listed above. The substrate material can also be a semiconductor (e.g., silicon). The broad range of substrate materials that can be utilized is due, in part, the ability to form nanostructured regions on the substrate at or near ambient temperatures or to the ability to attach previously formed nanostructured regions to the substrate.

According to various aspects of the invention, biologically active agents are disposed on and/or within a range of nanostructured regions, including nanoporous regions and nanotextured regions.

As noted above, biologically active agents are loaded in accordance with the present invention for any of a number of purposes, for example, to effect in vivo release of the biologically active agents (which may be, for example, immediate or sustained release), to influence (e.g., either promote or inhibit) bonding between the medical device and adjacent tissue, to influence thromboresistance, to influence antihyperplastic behavior, to enhance recellularization, and to promote tissue neogenesis, among many other purposes.

The medical devices of the present invention can be loaded with biologically active agents such the biologically active agents are released, retained or both upon contact with a patient.

For example, in embodiments where tortuous paths are created by an interconnected nanoporous network and/or where pore diameters approach the size of the agent to be delivered, release of biologically active agents can be significantly delayed, in some instances approaching zero order release kinetics.

As another example, in embodiments where surface features associated with nanostructured regions are filled with biologically active agents that are retained upon patient contact, nano-sized areas of the biologically active agents are created in some instances to control cellular interactions and adhesion.

In some embodiments, a first biological agent (e.g., a glycosaminoglycan) can also act as a reservoir for an additional biologically active agent, (e.g., an endogenous growth factor).

Examples of biologically active agents that control (e.g., promote or inhibit) cell growth and/or attachment to the medical devices of the present invention include polysaccharides such as glycosaminoglycans and proteoglycans, for example, hyaluronic acid (e.g., to inhibit tissue adhesion), keratan, perlecan, dermatin, heparin and chondroitin, as well as various salts of the same, such as hyaluronates, dermatin sulfates, heparin sulfates, keratan sulfates and chondroitin sulfates; cell adhesion peptides (e.g., RGD peptides); adhesive proteins (e.g., fibronectin, laminin, vitronectin, etc.); and growth factors. Synthetic materials also can be used to control biologic reactions and can have biologic activity as well. For example, sulfonated polymers can act as synthetic heparinoids, and synthetic hydrogels (e.g., PEG) can act as anti-adhesives. Numerous additional biologically active agents are presented below.

As noted above, nanostructured regions (including nanoporous regions and nanotextured surface regions), whether with or without biologically active agents, can correspond to the entire medical device surface, or to only a portion (or portions) of the medical device. Hence, one or more nanostructured regions can be provided on the medical device surface at desired locations and/or in desired shapes (e.g., in desired patterns, for instance, using appropriate masking techniques, including lithographic techniques). For example, for tubular devices such as stents (which can comprise, for example, a laser or mechanically cut tube, one or more braided, woven, or knitted filaments, etc), the nanostructured regions can be provided on the luminal surfaces, on the abluminal surfaces, on the lateral surfaces between the luminal and abluminal surfaces, patterned along the luminal or abluminal length of the devices, on the ends, and so forth. Moreover, multiple nanostructured regions can be formed using the same or different techniques, and can contain the same biologically active agent, different biologically active agents, or no biologically active agent. It is therefore possible, for example, to release the same or different therapeutic agents at different rates from different locations on the medical device. As another example, it is possible to provide a tubular tubular medical device (e.g., a vascular stent) having a first nanoporous region comprising a first biologically active agent (e.g., an antithrombotic agent) on its inner, luminal surface and a second nanoporous region comprising a second biologically active agent that differs from the first biologically active agent (e.g., an antiproliferative agent) on its outer, abluminal surface (as well as on the ends).

Where utilized, biologically active agents can be associated with nanostructured regions using a variety of techniques. For example, as discussed elsewhere herein, in some embodiments, the biologically active agents are incorporated concurrently with the formation of the nanostructured regions. In other instances the biologically active agents are incorporated subsequent to the formation of the nanostructured regions.

For example, in some embodiments, a fluid containing dissolved or dispersed biologically active agent is contacted with a nanostructured region, for instance, by spray coating, physical application (e.g., by rolling or brushing), spin-coating and immersion, among other techniques. Water, organic solvents, subcritical fluids, critical point fluids, supercritical fluids, and so forth can be used as carriers for the biologically active agent. (The use of supercritical fluids to load nanoporous regions with biologically active agent is described, for example, in U.S. patent application Ser. No. ______, entitled “Use of Supercritical Fluids to Incorporate Biologically Active Agents into Nanoporous Medical Articles,” Attorney Docket No. 03-084, filed on even date herewith.) Moreover, methods such as lyophilization, or exposure to critical point solutions or supercritical fluids, are optionally employed to remove any residual solvent, where appropriate.

In some embodiments, pores are further filled with sol-gels in order to control biologic interactions, including sol-gels based on bioactive ceramics such as those discussed above.

The present invention is applicable to a wide variety of medical devices including controlled drug delivery devices and other medical devices. Medical devices for use in conjunction with the various embodiments of the present invention include devices that are implanted or inserted into the body, either for procedural uses or as implants. Examples of medical devices for use in conjunction with the present invention include orthopedic prosthesis such as bone grafts, bone plates, joint prostheses, central venous catheters, vascular access ports, cannulae, metal wire ligatures, stents (including coronary vascular stents, cerebral, urethral, ureteral, biliary, tracheal, gastrointestinal and esophageal stents), stent grafts, vascular grafts, catheters (for example, renal or vascular catheters such as balloon catheters), guide wires, balloons, filters (e.g., vena cava filters), tissue scaffolding devices, tissue bulking devices, embolization devices including cerebral aneurysm filler coils (e.g., Guglilmi detachable coils and metal coils), heart valves, left ventricular assist hearts and pumps, and total artificial hearts.

Metallic nanostructured surfaces can also provided on various electrodes, including neural electrodes (e.g., for ocular and otological implants and for muscle stimulation in paraplegics), pacemaker electrodes, and ablation electrodes (e.g., cardiac ablation devices), for example, to increase the surface area and effective charge density associated with the same.

The medical devices of the present invention may be used for systemic treatment or for localized treatment of any mammalian tissue or organ. Examples are tumors; organs including but not limited to the heart, coronary and peripheral vascular system (referred to overall as “the vasculature”), lungs, trachea, esophagus, brain, liver, kidney, bladder, urethra and ureters, eye, intestines, stomach, pancreas, ovary, and prostate; skeletal muscle; smooth muscle; breast; cartilage; and bone.

As used herein, “treatment” refers to the prevention of a disease or condition, the reduction or elimination of symptoms associated with a disease or condition, or the substantial or complete elimination a disease or condition. Preferred subjects (also referred to as “patients”) are vertebrate subjects, more preferably mammalian subjects and more preferably human subjects.

“Biologically active agents,” “drugs,” “therapeutic agents,” “pharmaceutically active agents,” “pharmaceutically active materials,” and other related terms may be used interchangeably herein and include genetic biologically active agents, non-genetic biologically active agents and cells. Biologically active agents may be used singly or in combination. Where used in combination, one biologically active agent may provide a matrix for another biologically active agent. A wide variety of biologically active agents can be employed in conjunction with the present invention. Numerous biologically active agents, not necessarily exclusive to those previously discussed, are described here.

Exemplary non-genetic biologically active agents for use in connection with the present invention include: (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, antimicrobial peptides such as magainins, aminoglycosides and nitrofurantoin; (m) cytotoxic agents, cytostatic agents and cell proliferation affectors; (n) vasodilating agents; (o) agents that interfere with endogenous vasoactive mechanisms, (p) inhibitors of leukocyte recruitment, such as monoclonal antibodies; (q) cytokines; and (r) hormones. Preferred non-genetic biologically active agents include paclitaxel, sirolimus, everolimus, tacrolimus, dexamethasone, estradiol, ABT-578 (Abbott Laboratories), trapidil, liprostin, Actinomcin D, Resten-NG, Ap-17, abciximab, clopidogrel and Ridogrel.

Exemplary genetic biologically active agents for use in connection with the present invention include anti-sense DNA and RNA as well as DNA coding for: (a) anti-sense RNA, (b) tRNA or rRNA to replace defective or deficient endogenous molecules, (c) angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine, polyethyleneimine (PEI)), graft copolymers (e.g., polyether-PEI and polyethylene oxide-PEI), neutral polymers PVP, SP1017 (SUPRATEK), lipids such as cationic lipids, liposomes, lipoplexes, nanoparticles, or microparticles, with and without targeting sequences such as the protein transduction domain (PTD).

Cells for use in connection with the present invention include cells of human origin (autologous or allogeneic), including whole bone marrow, bone marrow derived mono-nuclear cells, progenitor cells (e.g., endothelial progenitor cells), stem cells (e.g., mesenchymal, hematopoietic, neuronal), pluripotent stem cells, fibroblasts, myoblasts, satellite cells, pericytes, cardiomyocytes, skeletal myocytes or macrophage, or from an animal, bacterial or fungal source (xenogeneic), which can be genetically engineered, if desired, to deliver proteins of interest.

Numerous biologically active agents, not necessarily exclusive of those listed above, have been identified as candidates for vascular treatment regimens, for example, as agents targeting restenosis. Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) ACE inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and O-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, fluvastatin, simvastatin and cerivastatin, (u) fish oils and omega-3fatty acids, (v) free-radical scavengers/antioxidants such as probucol, vitamins C and E, ebselen, trans-retinoic acid and SOD mimics, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) MMP pathway inhibitors such as marimastat, ilomastat and metastat, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, paclitaxel and epothilone), caspase activators, proteasome inhibitors, angiogenesis inhibitors (e.g., endostatin, angiostatin and squalamine), rapamycin, cerivastatin, flavopiridol and suramin, (aa) matrix deposition/organization pathway inhibitors such as halofuginone or other quinazolinone derivatives and tranilast, (bb) endothelialization facilitators such as VEGF and RGD peptide, and (cc) blood rheology modulators such as pentoxifylline.

Numerous additional biologically active agents useful for the practice of the present invention are also disclosed in U.S. Pat. No. 5,733,925 assigned to NeoRx Corporation, the entire disclosure of which is incorporated by reference.

A range of biologically active agent loading levels can be used in connection with the various embodiments of the present invention, with the amount of loading being readily determined by those of ordinary skill in the art and ultimately depending, for example, upon the condition being treated, the degree to which it is desired to influence cell adhesion, the nature of the biologically active agent, the means by which the biologically active agent is administered to the intended subject, and so forth.

Although various embodiments are specifically illustrated and described herein, it will be appreciated that modifications and variations of the present invention are covered by the above teachings and are within the purview of the appended claims without departing from the spirit and intended scope of the invention.

1. An implantable or insertable medical device comprising (a) a nanoporous region that comprises interconnected nanopores and (b) a biologically active agent disposed within said interconnected nanopores of said nanoporous region, wherein the lateral dimensions of said nanopores approach the hydrated radius of said biologically active agent.

2. The implantable or insertable medical device of claim 1, wherein said biologically active agent is a cell-adhesion promoting biomolecule.

3. The implantable or insertable medical device of claim 2, wherein said cell-adhesion promoting biomolecule is selected from glycosaminoglycans, proteoglycans, cell adhesion peptides, and adhesive proteins.

4. The implantable or insertable medical device of claim 1, wherein said nanoporous region is (a) provided in the shape of said medical device or a component of said medical device or (b) provided on a substrate that corresponds to said medical device or a component of said medical device.

5. The implantable or insertable medical device of claim 1, wherein said medical device comprises two or more nanoporous regions.

6. An implantable or insertable medical device comprising (a) a metallic or ceramic nanoporous region that comprises interconnected nanopores and (b) a biologically active agent disposed within said interconnected nanopores of said nanoporous region, wherein said biologically active agent is established within said nanoporous region concurrently with the formation of said nanoporous region and at temperatures below the degradation temperature of the biologically active agent.

7. The implantable or insertable medical device of claim 6, wherein said nanoporous region is formed at temperatures less than 100° C.

8. The implantable or insertable medical device of claim 6, wherein said biologically active agent is released from said interconnected nanopores upon implantation or insertion of said nanoporous region into a patient.

9. The implantable or insertable medical device of claim 6, wherein said biologically active agent is a cell-adhesion promoting biomolecule.

10. The implantable or insertable medical device of claim 6, wherein said nanoporous region is (a) provided in the shape of said medical device or a component of said medical device or (b) provided on a substrate that corresponds to said medical device or a component of said medical device.

11. The implantable or insertable medical device of claim 6, wherein the lateral dimensions of said interconnected nanopores approach the hydrated radius of the biologically active agent.

12. The implantable or insertable medical device of claim 6, wherein said medical device comprises two or more nanoporous regions.

13. An implantable or insertable medical device comprising a nanoporous region, said nanoporous region being formed by a method that comprises: (a) providing a precursor region comprising a first material that is present in nano-domains within said precursor region; and (b) subjecting said precursor region to conditions under which said first material is either reduced in volume or eliminated from said precursor region, thereby forming a nanoporous region.

14. The implantable or insertable medical device of claim 13, further comprising a biologically active agent disposed within said nanoporous region.

15. The implantable or insertable medical device of claim 14, wherein said biologically active agent is a cell-adhesion promoting biomolecule.

16. The implantable or insertable medical device of claim 13, wherein the lateral dimensions of the nanopores formed within the nanoporous region approach the hydrated radius of the biologically active agent.

17. The implantable or insertable medical device of claim 13, wherein said nanoporous region is (a) provided in the shape of said medical device or a component of said medical device or (b) provided on a substrate that corresponds to said medical device or a component of said medical device.

18. The implantable or insertable medical device of claim 13, wherein said medical device comprises two or more nanoporous regions.

19. The implantable or insertable medical device of claim 13, wherein said precursor region comprises sinterable nanoparticles and evanescent nanoparticles, and wherein said precursor region is heated such that at least a portion of the sinterable nanoparticles are sintered and at least a portion of the evanescent nanoparticles are converted to vapor, thereby forming said nanoporous region.

20. The implantable or insertable medical device of claim 19, wherein said sinterable nanoparticles are metal nanoparticles

21. The implantable or insertable medical device of claim 19, wherein said sinterable nanoparticles are ceramic nanoparticles.

22. The implantable or insertable medical device of claim 19, wherein said sinterable nanoparticles are bioactive ceramic nanoparticles.

23. The implantable or insertable medical device of claim 19, wherein said sinterable nanoparticles are polymer nanoparticles.

24. The implantable or insertable medical device of claim 19, wherein said evanescent nanoparticles are combustible nanoparticles.

25. The implantable or insertable medical device of claim 19, wherein said evanescent nanoparticles are evaporable nanoparticles.

26. The implantable or insertable medical device of claim 19, wherein said evanescent nanoparticles are sublimable nanoparticles.

27. The implantable or insertable medical device of claim 26, wherein said sublimable nanoparticles comprise a sublimable metal selected from calcium and magnesium.

28. The implantable or insertable medical device of claim 26, wherein said sublimable nanoparticles comprise a sublimable organic compound.

29. The implantable or insertable medical device of claim 19, wherein said evanescent nanoparticles comprise a reducible oxide of a sublimable metal, and wherein said mixture is heated under a reducing atmosphere.

30. The implantable or insertable medical device of claim 19, wherein said nanoporous region comprises a gradient in pore volume.

31. The implantable or insertable medical device of claim 13, wherein said precursor region comprises sinterable nanoparticles and oxide nanoparticles, and wherein said precursor region is heated under reducing conditions such that at least a portion of the sinterable nanoparticles are sintered and at least a portion said reducible oxide nanoparticles are reduced, thereby decreasing the volume of said oxide nanoparticles and establishing said nanoporous region.

32. The implantable or insertable medical device of claim 13, wherein said precursor region is an alloy comprising a plurality of metals of differing nobility; and wherein at least a portion of a less-noble metal within said alloy is removed to form said nanoporous region.

33. The implantable or insertable medical device of claim 32, wherein said alloy comprises gold and silver, and wherein silver is oxidized and removed.

34. The implantable or insertable medical device of claim 32, wherein said less-noble metal is removed by a process selected from (a) contacting the alloy with a solution having an acidity effective to oxidize said less-noble metal; (b) immersing said alloy in an electrolyte and applying a voltage of a magnitude and bias effective to oxidize said less-noble metal; and (c) heating said alloy in the presence of oxygen to a temperature that is effective to oxidize said less-noble metal.

35. An implantable or insertable medical device comprising a nanostructured region, said nanostructured region provided by a method that comprises one or more of the following processes: (a) a physical vapor deposition process comprising evaporation of a metal oxide, (b) a physical vapor deposition process comprising sublimation of a metal or ceramic material, (c) a physical vapor deposition process comprising sputtering of a metal or metal oxide, (d) a physical vapor deposition process comprising laser ablation of a metal or ceramic material, (e) simultaneous physical vapor deposition of (i) a metal or a ceramic material and (ii) a biologically active agent, (f) ion deposition of a metal or metal oxide layer, (h) ion implantation into a metal or ceramic surface, (i) X-ray lithography of a metal or ceramic surface, (j) a kinetic metallization process, (k) chemical vapor deposition of a metal or ceramic material, (l) electrodeposition and (m) electroless deposition.

36. The implantable or insertable medical device of claim 35, wherein said nanostructured region is a nanotextured region.

37. The implantable or insertable medical device of claim 35, wherein said nanostructured region is a nanoporous region.

38. The implantable or insertable medical device of claim 35, wherein said nanostructured region comprises a biologically active agent.

39. The implantable or insertable medical device of claim 38, wherein said biologically active agent is a cell-adhesion promoting biomolecule.

40. The implantable or insertable medical device of claim 38, wherein said biologically active agent is released from said nanostructured region upon implantation or insertion of said nanoporous region into a patient.

41. The implantable or insertable medical device of claim 37, wherein said nanoporous region comprises a biologically active agent.

42. The implantable or insertable medical device of claim 41, wherein the lateral dimensions of nanopores in said nanoporous region approach the hydrated radius of the biologically active agent.

43. The implantable or insertable medical device of claim 41, wherein said biologically active agent is established within said nanoporous region concurrently with the formation of said nanoporous region.

44. The implantable or insertable medical device of claim 35, wherein said medical device comprises a plurality of distinct nanoporous regions.

45. The implantable or insertable medical device of claim 35, wherein said nanostructured region comprises a material that is present in nano-domains within said nanostructured region; and wherein said nanostructured region is subjected to conditions under which said material is either reduced in volume or eliminated from said precursor region, thereby forming a nanoporous region.

46. The implantable or insertable medical device of claim 35, wherein said nanostructured region is provided by a method in which inert ions are ionized and accelerated into the surface region during physical vapor deposition.

47. The implantable or insertable medical device of claim 35, wherein said nanostructured region is provided by a method in which at least a portion of the deposited material or a precursor thereof is ionized and accelerated to the surface region during deposition.

48. The implantable or insertable medical device of claim 35, wherein said nanostructured region is provided by a method that comprises an ion implantation process and wherein said implanted ion is an inert ion.

49. The implantable or insertable medical device of claim 35, wherein said nanostructured region is provided by a method that comprises an ion implantation process and wherein said implanted ion is a reactive ion.

50. The implantable or insertable medical device of claim 35, wherein said nanostructured region is provided by a method that comprises an ion implantation process and wherein said implanted ion corresponds to an element or molecule of the surface region into which it is implanted.

51. The implantable or insertable medical device of claim 35, wherein said nanostructured region is provided by a method that comprises a kinetic metallization process and wherein said kinetic metallization process comprises impacting a substrate with two or more metal particle populations, each of different compositions.

52. The implantable or insertable medical device of claim 35, wherein said nanostructured region is provided by a method that comprises a kinetic metallization process and wherein said kinetic metallization process comprises concurrently impacting a substrate with metal particles and with a biologically active agent.

53. The implantable or insertable medical device of claim 35, wherein said nanostructured region is provided by a method that comprises a chemical vapor deposition process and wherein said chemical vapor deposition process is particle-precipitation-aided chemical vapor deposition process.

54. The implantable or insertable medical device of claim 35, wherein said nanostructured region is formed directly on a substrate that corresponds to said medical device or a component of said medical device.

55. The implantable or insertable medical device of claim 35, wherein said nanostructured region is applied after formation to a substrate that corresponds to said medical device or a component of said medical device.

56. An implantable or insertable medical device comprising a nanotextured surface region and a cell-adhesion-promoting biomolecule provided on, within or beneath said nanotextured surface region.

57. The implantable or insertable medical device of claim 56, wherein said cell-adhesion-promoting biomolecule is selected from glycosaminoglycans, proteoglycans, cell adhesion peptides, and adhesive proteins.

58. The implantable or insertable medical device of claim 35, wherein said nanostructured region is provided by a method that comprises a electrodeposition or electroless deposition process wherein said electrodeposition or electroless deposition process comprises concurrently depositing two or more materials, each of different composition, wherein one or more said materials can be biologically active agents.

59. The implantable or insertable medical device of claim 1, wherein said device is a tubular medical device that comprises a nanoporous region comprising a first biologically active agent on its inner luminal surface and a nanoporous region comprising a second biologically active agent that differs from said first biologically active agent on its outer abluminal surface.

60. The implantable or insertable medical device of claim 59, wherein said device is a vascular stent and wherein said first biologically active agent is an antithrombotic agent and wherein said second biologically active agent is an antiproliferative agent.

61. The implantable or insertable medical device of claim 6, wherein said device is a tubular medical device that comprises a nanoporous region comprising a first biologically active agent on its inner luminal surface and a nanoporous region comprising a second biologically active agent that differs from said first biologically active agent on its outer abluminal surface.

62. The implantable or insertable medical device of claim 61, wherein said device is a vascular stent and wherein said first biologically active agent is an antithrombotic agent and wherein said second biologically active agent is an antiproliferative agent.

63. The implantable or insertable medical device of claim 14, wherein said device is a tubular medical device that comprises a nanoporous region comprising a first biologically active agent on its inner luminal surface and a nanoporous region comprising a second biologically active agent that differs from said first biologically active agent on its outer abluminal surface.

64. The implantable or insertable medical device of claim 63, wherein said device is a vascular stent and wherein said first biologically active agent is an antithrombotic agent and wherein said second biologically active agent is an antiproliferative agent.

65. The implantable or insertable medical device of claim 38, wherein said device is a tubular medical device that comprises a nanoporous region comprising a first biologically active agent on its inner luminal surface and a nanoporous region comprising a second biologically active agent that differs from said first biologically active agent on its outer abluminal surface.

66. The implantable or insertable medical device of claim 65, wherein said device is a vascular stent and wherein said first biologically active agent is an antithrombotic agent and wherein said second biologically active agent is an antiproliferative agent.

67. The medical device of claim 1, wherein said nanoporous region is a patterned nanoporous region.

68. The medical device of claim 1, wherein said device is an implantable or insertable tubular medical device, and wherein the nanoporous region is provided only on the inner luminal surface of the device, only on the outer abluminal surface of the device, or only on the edges between the luminal and abluminal surfaces of the device.

69. The medical device of claim 6, wherein said nanoporous region is a patterned nanoporous region.

70. The medical device of claim 6, wherein said device is an implantable or insertable tubular medical device, and wherein the nanoporous region is provided only on the inner luminal surface of the device, only on the outer abluminal surface of the device, or only on the edges between the luminal and abluminal surfaces of the device.

71. The medical device of claim 13, wherein said nanoporous region is a patterned nanoporous region.

72. The medical device of claim 13, wherein said device is an implantable or insertable tubular medical device, and wherein the nanoporous region is provided only on the inner luminal surface of the device, only on the outer abluminal surface of the device, or only on the edges between the luminal and abluminal surfaces of the device.

73. The medical device of claim 35, wherein said nanostructured region is a patterned nanostructured region.

74. The medical device of claim 35, wherein said device is an implantable or insertable tubular medical device, and wherein the nanostructured region is provided only on the inner luminal surface of the device, only on the outer abluminal surface of the device, or only on the edges between the luminal and abluminal surfaces of the device.

75. The medical device of claim 56, wherein said nanotextured surface region is a patterned nanotextured surface region.

76. The medical device of claim 56, wherein said device is an implantable or insertable tubular medical device, and wherein the nanotextured surface region is provided only on the inner luminal surface of the device, only on the outer abluminal surface of the device, or only on the edges between the luminal and abluminal surfaces of the device.
